(19) United States
(12) Patent Application Publication
(10) Pub. No.: US 2026/0166327 A1
(43) Pub. Date: Jun. 18, 2026

(54) LEADLESS IMPLANTABLE MEDICAL DEVICES

(71) Applicant: GEORGIA STATE UNIVERSITY RESEARCH FOUNDATION, INC., Atlanta, GA (US)

(72) Inventors: Sidong LEI, Atlanta, GA (US); Tara JABEGU, Atlanta, GA (US); Diren MARABA, Atlanta, GA (US)

(21) Appl. No.: 18/875,299

(22) PCT Filed: Jun. 16, 2023
(86) PCT No.: PCT/US2023/068562

§ 371 Date: Dec. 16, 2024

Related U.S. Application Data
(60) Provisional application No. 63/353,210, filed on Jun. 17, 2022.

(51) Int. Cl. A61N 1/375

(52) U.S. Cl. CPC A61N 1/3756; A61N 1/37512; A61N 1/37518

(57) ABSTRACT

Described herein are leadless implantable devices. An example device includes a first body portion, which includes a shell, a housing configured to house at least one electrical component, the housing being arranged at least partially within the shell, and a coupler attached to the housing. The device also includes a second body portion configured to anchor to a surface of a patient's heart. Additionally, the coupler includes a base and a plurality of projecting members extending from the base, and the coupler is configured to detachably couple to the second body portion.

### CROSS-REFERENCE TO RELATED APPLICATIONS

This application claims the benefit of U.S. provisional patent application No. 63/353,210, filed on Jun. 17, 2022, and titled “LEADLESS IMPLANTABLE MEDICAL DEVICES,” the disclosure of which is expressly incorporated herein by reference in its entirety.

### BACKGROUND

According to the World Health Organization (WHO), around one third of the worldwide deaths are due to cardiovascular diseases (CVDs) and CVDs are the leading cause of death globally. The death percentage increase during 2007-2017 period by 21.1% indicating the ever increasing deaths due to CVDs [1]. Developing new devices, techniques, and medicines for any type of CVDs are essential for reducing this large number of deaths due to CVDs.

Heart chambers have a certain rhythm for contraction and relaxation to ensure the perfect flow of blood in the human body. It is done by the cardiac muscles of the heart with the help of electric signals generated by the nervous system at the sinoatrial node. Voltage-clamp methods were able to study the ionic mechanism of the rhythmic contraction and relaxation of the cardiac membranes responsible for pacemaking [2, 3]. Rhythmicity of the heart comes from the spontaneous movement of the sinoatrial node of pacemaker cells of mammals, their typical action potential, and slow diastolic depolarization. The action potential is the peak potential of the electric signal generated at the sinoatrial node, and it is responsible for the systolic contraction of the cardiac muscles. The slow depolarizing signal just after the peak potential is the diastole of the heart, which is responsible for the relaxation of the heart. This slowly depolarizing signal plays the primary role in pacemaking. Due to different causes like bradyarrhythmias, tachyarrhythmias, and conduction abnormalities, this process goes out of perfect periodicity [4]. For the correction of these problems in the human heart, the first pacemaker device was developed and implanted in a human in 1958 [5]. Since then, many pacemaker devices have been developed from single chamber asynchronous pacemakers [5] to dual-chamber programmable synchronous pacemakers [6] and, more lately, leadless pacemakers (LPMs) as well [7, 8].

Traditional pacemakers are very uncomfortable since they are large in size and contain very long electrodes. The main part containing electronics and battery is placed under the skin at the chest area, and two or more electrodes are passed through veins to the heart chambers from the main part. In contrast, the latest LPMs like transcatheter pacing system (TPS) (e.g., the MICRA transcatheter pacing system from Medtronic plc of Dublin, Ireland) and leadless cardiac pacemakers (LCP) (e.g., the NANOSTIM pacemaker system of St. Jude Medical Inc. of St. Paul, MN), are very small, around 26-42 mm in length, just 1 cm 3 in volume, and very lightweight of only 2 g versus 21.5 g of traditional pacemakers [7, 8]. These LPMs are directly placed inside the heart chambers, and hence they do not require long electrodes. These LPMs not only solved the issue of bulkiness and uncomfortableness of traditional pacemakers but also have a longer battery life of 7-15 years due to the miniaturization of the electronics [9]. Although LPM systems are in the early stage of development and have not yet reached their full life period since early implantations, several comparative studies between LCP, TPS, and traditional transvenous pacemakers prove the superiority of leadless pacemakers [10, 11]. Albeit their superiority to traditional pacemakers, LPMs still suffer problems like pericardial effusions, vascular complications, cardiac perforation, venous thromboembolism, dislodgement into the pulmonary arteries, etc. [12].

One major problem arising from the long-term use of leadless pacemaking systems is their afterlife management. This is currently done either by turning the device off remotely and leaving it inside the heart forever or by extracting the device out of the patient's body [13]. This is indispensable because sometimes a component of the device fails, or the battery drains out at the end. When devices are just turned off, multiple devices will remain within a single chamber of the heart, which is medically undesirable. Extraction of the device from the heart, on the other hand, also is unsafe and not simple. Failures of the extraction process were reported in pre-clinical [14] to clinical stages as well [15]. The extraction causes severe damage to cardiac tissues [16]. In turn, a one-month bridge period for replacement of a leadless pacemaker is also suggested for tissue recovery for better implantation of the next pacemaker [17].

Therefore additional devices and systems that address these and other challenges associated with LPMs are needed.

### SUMMARY

An example leadless implantable device can include a first body portion, the first body portion including a shell and a housing. The housing configured to house at least one electrical component, the housing being arranged at least partially within the shell, and a coupler attached to the housing; and a second body portion configured to anchor to a surface of a patient's heart, where the coupler includes a base and a plurality of projecting members extending from the base, and where the coupler is configured to detachably couple to the second body portion.

In some implementations, the coupler is configured to detachably couple to the second body portion at any face-to-face angle between the base of the coupler and the second body portion. Alternatively or additionally, in some implementations the coupler is configured to detachably couple to the second body portion with minimal force.

In some implementations, the coupler includes about five projecting members. Optionally, each of the projecting members is an elongate member. In some implementations, the elongate member is about 5 millimeters (mm) long by 2 mm wide.

In some implementations, the shell is moveable relative to the housing between a first position and a second position. Alternatively or additionally, the first body portion further includes an actuation component configured to return the shell from the second position to the first position. Optionally, the actuation component is a spring. Alternatively or additionally, the projecting members are configured to retract inside the shell when the shell is disposed in the first position.

In some implementations, the projecting members are configured to extend outside of the shell when the shell is disposed in the second position. Optionally, the projecting members are configured to extend beyond a perimeter of the shell when the shell is disposed in the second position. Alternatively or additionally, the coupler is configured to mechanically couple the first body portion to the second body portion. Optionally, the shell is configured to prevent the coupler from mechanically uncoupling the first body portion from the second body portion when the shell is disposed in the first position.

In some implementations, the coupler is configured to provide electrical coupling between the first and second body portions. Optionally, a portion of the coupler is conductive and a portion of the second body portion is conductive. Alternatively or additionally, the conductive portion of the coupler is one or more of the projecting members. Optionally, the conductive portion of the coupler is at least a portion of the base.

In some implementations, each of the projecting members includes a respective boss. Optionally, each of the projecting members defines a respective first end and a respective second end opposite to the respective first end, where the respective first end is arranged in proximity to the base, and where the respective boss is arranged in proximity to the respective second end. Optionally, the respective bosses are configured to engage with a corresponding void in the second body portion.

In some implementations, each of the projecting members is formed from a plastic material having a conductive coating.

In some implementations, each of the projecting members is formed from a metal.

In some implementations, the at least one electrical component includes a power source, a pulse generator configured to deliver electrical stimulation, or a controller including a processor and a memory.

In some implementations, one or more of the projecting members is a flexible projecting member.

Additionally, a system including the leadless implantable device described above a control device configured to mechanically actuate the first body portion of the leadless implantable device is described herein. Optionally, the control device includes a motor, a gear system mechanically coupled to the motor, and a plurality of control arms mechanically coupled to the gear system. The control arms are configured to detachably couple to the shell of the first body portion of the leadless implantable device.

An example leadless implantable device can include a shell; a housing configured to house at least one electrical component, the housing being arranged at least partially within the shell; and a coupler attached to the housing, where the coupler includes a base and a plurality of projecting members extending from the base. Optionally, the coupler is configured to detachably couple to an anchor attached to a surface of a patient's heart.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIGS. 1A-1D illustrate a leadless implantable device according to implementations described herein. FIG. 1A illustrates a controller (sometimes referred to herein as “control device”), a main DLPM (detachable leadless pacemaker) body (sometimes referred to herein as “first body portion”), and hook (sometimes referred to herein as “second body portion”) of a leadless pacemaker system according to one implementation of the present disclosure. FIGS. 1B-1D illustrate attachment between the main DLPM body and the hook according to one implementation of the present disclosure. FIG. 1B illustrates the two parts separately (relaxed condition), FIG. 1C illustrates the main DLPM body in a state ready for attachment to the hook (attachment ready condition), and FIG. 1D illustrates the attached configuration between the main DLPM body and the hook (attached condition).

FIGS. 2A-2D are images of a DLPM according to an implementation of the present disclosure. FIG. 2A is a rendered image which shows the inner hollow capsule with top electrode, spring and claws at bottom. FIG. 2B is a rendered image of an outer shell. FIG. 2C is a rendered image of an assembled device. FIG. 2D is an optical image of an assembled device.

FIGS. 3A-3D illustrate the operation of a DLPM according to an implementation of the present disclosure. FIG. 3A is a schematic image of a planetary gear that can be used in implementations of the present disclosure. FIG. 3B illustrates a released condition of the controller. FIG. 3C illustrates the controller grabbing the DLPM with its arms. FIG. 3D illustrates the state of the device after the controller has pulled the shell of the DLPM, opening the claws wide open.

FIGS. 4A-4D illustrate a demonstration of DLPM implant and replacement according to an implementation of the present disclosure. FIG. 4A illustrates an artificial heart. FIG. 4B illustrates a first DLPM inside the artificial heart. FIG. 4C illustrates the hook after the extraction of the first DLPM.

FIG. 4D illustrates the new replaced DLPM attached to the same hook shown in FIG. 4C.

FIGS. 5A-5B illustrate pulse signal measurements from the DLPM of FIGS. 4A-4D. FIG. 5A illustrates the experimental setup for pulse signal measurements. FIG. 5B illustrates a graph of the pulse signal measured.

FIG. 6 illustrates the implantation and removal of a leadless implantable device according to one implementations of the present disclosure.

FIG. 7 is an example computing device.

FIG. 8 is a circuit diagram of example electronic components of a DLPM according to an implementation of the present disclosure.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for implantable pacemakers, it will become evident to those skilled in the art that the implementations are not limited thereto, but are applicable for other implantable devices.

As used herein, the terms “about” or “approximately” when referring to a measurable value such as an amount, a percentage, and the like, is meant to encompass variations of ±20%, +10%, +5%, or +1% from the measurable value.

The term “subject” or “patient” is defined herein to include animals such as mammals, including, but not limited to, primates (e.g., humans), cows, sheep, goats, horses, dogs, cats, rabbits, rats, mice and the like. In some embodiments, the subject is a human.

Implementations of the present disclosure are directed to a detachable leadless pacemaker (DLPM) capable of regularizing the abnormal pacing of the heart. The DLPM described herein is able to eradicate the issues of afterlife management of leadless pacemakers (LPMs). Additionally, the DLPM described herein can overcome one or more issues associated with LPMs as described above, for example, because of the DLPM's detachable functionality. As described below, in some implementations, the DLPM has a hook that will be permanently hooked to the myocardium of the heart, and another detachable main part containing electronics and battery. This enables extraction of the dead pacemaker and replacement with the new one with the help of a controller very easily like a plug and play device.

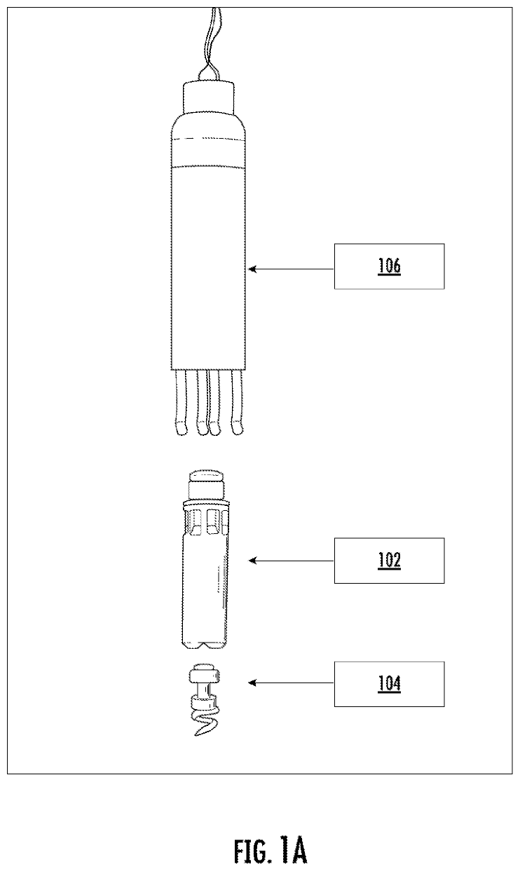

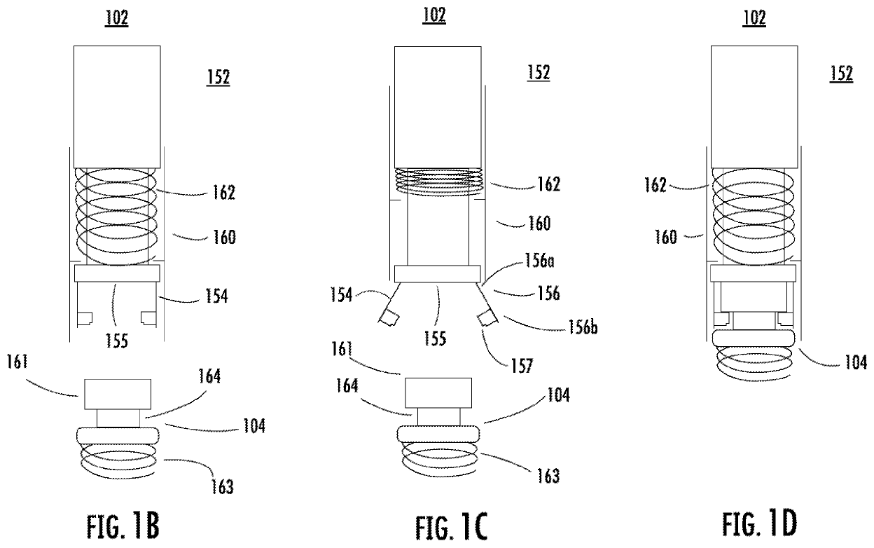

Referring now to FIGS. 1A-1D, a leadless implantable device according to implementations of the present disclosure is shown. The leadless implantable device can include a first body portion 102 (also referred to herein as a “detachable leadless pacemaker” or DLPM body) and a second body portion 104 (also referred to herein as a “hook”). As described herein, the second body portion 104 is configured to anchor to a surface of the subject's heart (e.g., attached to tissue within a chamber of the heart). In FIGS. 1B and 1C, the second body portion 104 is shown as a cylindrical body 161 having a spiral structure 163 with a sharp needle at its distal end. However, it should be understood and appreciated that the present disclosure is not limited only to implementations in which the second body portion 104 includes a spiral structure or spiral “hook”; rather, the second body portion 104 may include other structures or configurations of structures (not shown) for anchoring to a surface of the subject's heart.

As described herein, the spiral structure 163 is configured to hook (e.g., by twisting the second body portion 104) into myocardium of the subject's heart chamber such that the second body portion 104 is anchored to a surface of the subject's heart. The second body portion 104 also defines a void 164 (or channel, groove, etc.) to which a coupler (described below) can be attached, which couples the first and second body portions 102, 104. This disclosure contemplates that the void 164 may be a single void (e.g., continuously arranged around the second body portion 104) or a plurality of voids (e.g., a number of voids arranged on the second body portion 104 and corresponding to a number of projecting members of the coupler). It should be understood that the geometry, size, and/or shape of the second body portion 104 is only provided as an example. This disclosure contemplates providing a second body portion having different geometry, size, and/or shape than as shown in FIGS. 1A-1D. The first body portion 102 can include a shell 160 (also referred to herein as the “outer shell”), a housing 152 (also referred to herein as the “inner shell”), and a coupler 154 (also referred to herein as the “claws”) attached to the housing 152. The housing 152 can be arranged at least partially within the shell 160. For example, the housing 152 is arranged at least partially within the shell in a relaxed condition (see FIG. 1B), an intermediate condition (see FIG. 1C), and an attached condition (see FIG. 1D). This disclosure contemplates that the leadless implantable device described herein may have a length and/or diameter smaller than conventional leadless pacemakers known in the art such as the NANOSTIM pacemaker system of St. Jude Medical Inc. of St. Paul, MN, which has dimensions of 42.0 mm length×5.9 mm diameter, or the MICRA transcatheter pacing system from Medtronic plc of Dublin, Ireland, which has dimensions of 25.9 mm length×6.7 mm diameter. For example, the leadless implantable device described herein may have a length less than about 42 mm and/or diameter less than 6.7 mm. Optionally, in one implementation, the leadless implantable device has a diameter of about 7 mm and a length of about 33 mm (first body portion 102) and about 39 mm (total of the first and second body portions 102 and 104). This disclosure contemplates providing leadless implantable devices having dimensions different than those provided as examples. It should be understood that the dimensions of the leadless implantable device depend on a number of factors including, but not limited to, materials and/or manufacturing techniques (e.g., machining, three-dimensional printing, etc.).

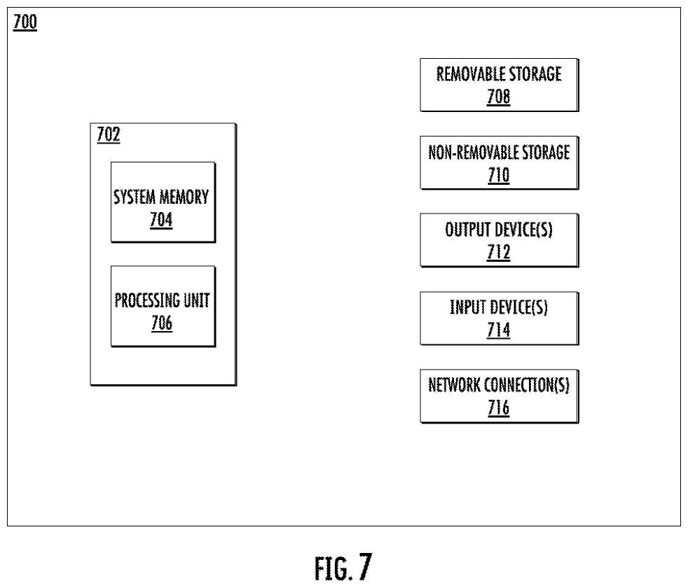

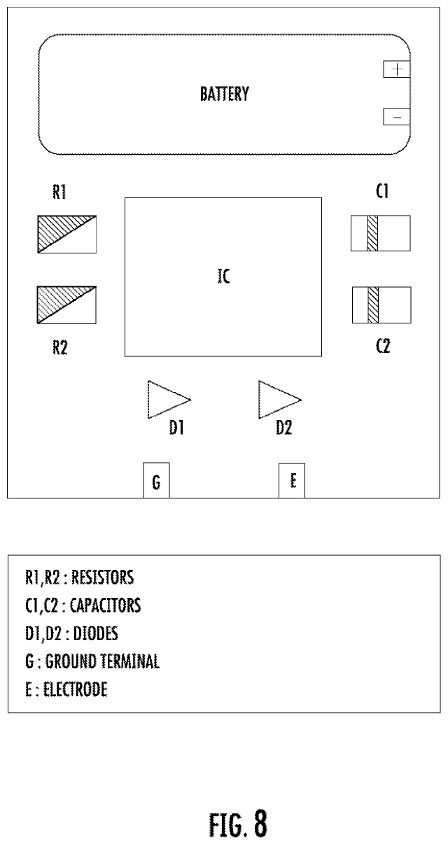

The housing 152 can be configured to house at least one electrical component (not shown in FIGS. 1A-1D). Such electrical components are the electronics required for pacing the subject's heart, including but not limited to, a power source (e.g., a battery), a pulse generator, and programmable logic (e.g., a processor and memory operably coupled to the processor such as the most basic configuration of example of FIG. 7). A circuit diagram of example electronic components are shown in FIG. 8. This disclosure contemplates that the pulse generator can be a voltage source or a current source. The pulse generator is configured to deliver electrical stimulation via one or more electrodes. Additionally, this disclosure contemplates that the programmable logic can be programmed to control operation of the pulse generator, for example, to regularize the abnormal pacing of the subject's heart. Pacemaker device electronics are well known in the art and therefore not described in further detail herein.

The coupler 154 can include a base 155 and a plurality of projecting members 156 extending from the base 155, where the coupler 154 is configured to detachably couple to the second body portion 104. Optionally, the projecting members 156 are flexible projecting members. The coupler 154 can detachably couple to the second body portion 104 mechanically with externally applied force and/or optionally with aid of another force (e.g., magnetic force). The present disclosure contemplates that different configurations of projecting members 156 are possible. Implementations of the present disclosure include a coupler 154 having five or six projecting members 156, which can be advantageous for maintaining electrical connection. As a non-limiting example, the coupler 154 can include about five projecting members 156. It should be understood that a coupler having five or six projecting members is provided only as an example. In other implementations, a coupler may have less than five projecting members, e.g., 2, 3, or 4 projecting members. In other implementations, a coupler may have more than five projecting members, e.g., 6, 7, 8, 9, etc. projecting members. This disclosure contemplates that ten to twelve projecting members may be a maximum number of projecting members for a coupler because adding more projecting members increases complexity without any gain in maintaining an electrical connection. Further, this disclosure contemplates that the coupler 154 may have only one (e.g., rather than a “plurality”) projecting member.

Alternatively or additionally, different shapes and sizes of projecting members 156 are contemplated. For example, this disclosure contemplates that the size and/or shape of projecting members 156 can be chosen based on the size of the device (e.g., first body portion 102 and second body portion 104), which includes consideration of the required opening angle for decoupling the coupler 154 from the second body portion 104. As non-limiting examples, each of the projecting members 156 can be an elongate member (i.e., a member having a length greater than width). Optionally, each elongate member can be about 5 millimeters (mm) long by about 2 millimeters (mm) wide. It should be understood that 5 mm length is provided only as an example. This disclosure contemplates elongate members having lengths greater or less than 5 mm. For example, the present disclosure contemplates that different ranges of opening are needed to decouple different sizes of elongate members from the second body portion. In one implementation, 5 mm long elongate members correspond to 30 degrees of opening for decoupling, and 4 mm long elongate members correspond to 45 degrees of opening for decoupling, which is more challenging to achieve. Accordingly, in this example implementation, 5 mm long elongate members would be preferred over 4 mm long elongate members. In some implementations, this disclosure contemplates a device having elongate members having a length of 5 mm ±1 mm. Additionally, it should be understood that 2 mm width is provided only as an example. This disclosure contemplates elongate members having widths greater or less than 2 mm. The elongate members can be flat in order to bend, and it should be understood that wider elongate members may require a larger shell body (e.g., shell 160 with a larger size). Accordingly, in this example implementation, 2 mm wide elongate members would be preferred over wider elongate members.

Alternatively or additionally, the coupler 154 can be configured to provide electrical coupling between the first body portion 102 and second body portion 104. For example, a portion of the coupler 154 can be conductive and a portion of the second body portion 104 can be conductive. The conductive portion of the coupler 154 can be one or more of the projecting members 156. For example, one or more of the projecting members 156 can be formed from a plastic material having a conductive coating in some implementations, or one or more of the projecting members 156 can be formed from a metal in other implementations. Alternatively or additionally, the conductive portion of the coupler 154 can be at least a portion of the base 155. For example, the base 155 can be formed from a plastic material having a conductive coating in some implementations, or the base 155 can be formed from a metal in other implementations. As described herein, the conductive portion of the coupler 154 can serve as the anode electrode for delivering electrical stimulation. Additionally, the conductive portion of the second body portion 104 can be a conductive surface of and/or the entire second body portion 104. For example, the second body portion 104 (or portion thereof) can be formed from a plastic material having a conductive coating in some implementations, or the second body portion 104 (or portion thereof) can be formed from a metal in other implementations. Accordingly, when the coupler 154 is mechanically coupled to the second body portion 104, there is an electoral connection between the first body portion 102 and second body portion 104. Thus, electrical stimulation, which is generated and delivered by the electrical component(s) contained within the housing 152, can be delivered to the subject's heart (e.g., pacemaking stimulation) via the second body portion 104, which is anchored to the subject's heart.

Optionally, in some implementations, each of the projecting members 156 can include a respective boss 157, which is illustrated in FIG. 1C. As used herein, a boss is a raised or protruding portion of the projecting member. For example, each of the projecting members 156 can define a respective first end 156a and a respective second end 156b opposite to the respective first end 156a, where the respective first end 156a is arranged in proximity to the base 155, and where the respective boss 157 is arranged in proximity to the respective second end 156b. This disclosure contemplates that the respective boss 157 can be arranged at the respective second end 156b (i.e., distal end of a projecting member) or spaced proximally from the respective second end 156b. The respective bosses 157 can be configured to engage with a corresponding void 164 in the second body portion 104. It should be understood that the geometry, size, and/or shape of the bosses 157 are provided only as examples. This disclosure contemplates providing bosses having different geometry, size, and/or shape than shown in FIG. 1C.

Additionally, in some implementations, the shell 160 can be movable relative to the housing 152 between a first position (“relaxed condition” of the device) and a second position (“attachment ready condition” of the device). The first position of the shell 160 is shown in FIG. 1B, and the second position of the shell 160 is shown in FIG. 1C. The first body portion 102 can also include an actuation component, which is illustrated as a spring 162 in FIGS. 1B-1C. The spring 162 is in an equilibrium state in FIG. 1B (relaxed condition), and the spring 162 is in a compressed state in FIG. 1C (attachment ready condition). Accordingly, the spring 162 is configured to return the shell 160 from the second position (see FIG. 1C) to the first position (see FIG. 1B), which is the relaxed condition of the device. It should be understood that a spring is only provided as an example actuation component and that the use of other actuation components is contemplated by the present disclosure. Alternative actuation components include, but are not limited to, rubber or other elastic components or material.

Additionally, as shown in FIG. 1B, the projecting members 156 are configured to be retracted inside the shell 160 when the shell 160 is disposed in the first position. In other words, in the relaxed condition of the device, the projecting members 156 are forced and maintained inside the shell 160. And as shown in FIG. 1C, the projecting members 156 are configured to extend outside the shell 160 when the shell 160 is disposed in the second position. In some implementations, the projecting members 156 are configured to extend beyond a perimeter of the shell 160 when the shell 160 is disposed in the second position (e.g. the position illustrated in FIG. 1C). In other words, in the attachment ready condition of the device, the projecting members 156 extend outside of the shell 160. This can be facilitated by a spring force of the projecting members 156. FIGS. 1B-1D illustrate a non-limiting example of these steps, where in FIG. 1B the coupler 154 is retracted inside the shell 160 (relaxed condition), and in FIG. 1C the coupler 154 is extended from the shell 160 (ready for attachment condition). In FIG. 1D, the coupler 154 is shown mechanically coupling the first body 102 portion to the second body portion 104. As shown in FIG. 1D, the shell 160 can be configured to prevent the coupler 154 from mechanically uncoupling the first body portion 102 from the second body portion 104 when the shell 160 is disposed in the first position (e.g. the position shown in FIG. 1D).

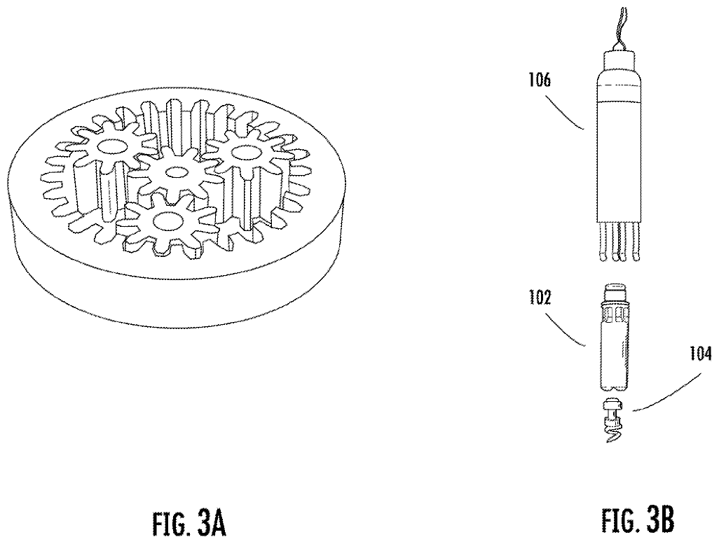

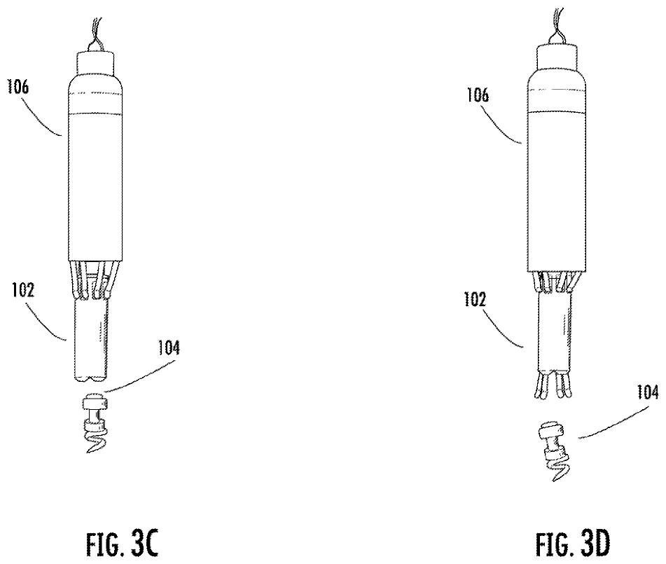

A system for implanting, removing, and/or replacing the leadless implantable device shown in FIGS. 1A-1D further includes a control device 106 configured to mechanically actuate the first body portion 102, which is shown in FIG. 1A. In particular, the control device 106 is configured to move the shell 160 between the first position (see FIG. 1B) and the second position (see FIG. 1C). This disclosure contemplates that the control device 106 can be attached to and delivered to the subject's heart using a flexible delivery tube (e.g., a catheter). For example, the control device 106 may be attached to the tip of the delivery tube for pacemaker installation or removal. The control device 106 can optionally include a motor, a gear system mechanically coupled to the motor, and a plurality of control arms mechanically coupled to the gear system. The control arms are configured to detachably couple to the shell 160. An example control device and operations are described in detail in the Examples below (see also FIGS. 3A-3D). For example, the motor can be configured to actuate, via the gear system, control arms of the control device 106 such that the control device performs grab-pull and push-release actions. From a relaxed condition (see FIGS. 1B and 3B), the control device 106 is placed in proximity to the first body portion 102 and energized to actuate the control arms to perform a grab-pull action. In this way, the control arms engage with the housing 152 and move the shell 160 from the first position (FIG. 1B) to the second position (FIG. 1C), which places the device in an attachment ready condition (see FIGS. 1C and 3D). In this condition, the projecting members 156 extend outside of the shell 160.

Additionally, as shown in FIG. 1C, when the shell 160 moves from the first position to the second position, a spring 162 is compressed. The control device 106 is then energized to actuate the control arms to perform a push-release action. In this way, the control arms release the housing 152 and the shell 160 moves from the second position (FIG. 1C) to the first position (FIG. 1B) due to the spring 162 returning to its uncompressed state. The actuation structure such as spring 162 therefore assists in returning the shell 160 to the first position. It should be understood that the control device 106 can be used to perform grab-pull and push-release actions to remove an existing device and replace with a new device. It should also be understood that a control device including a motor, a gear system, and a plurality of control arms (e.g., as discussed here and with reference to FIGS. 3A-3D) is provided only as an example. This disclosure contemplates using control devices having different configurations. For example, in some implementations, the control device includes a cable-driving mechanism (e.g., rather than a motor and gears, or in addition to a motor and gears) that includes flexible cables deliverer force to the first body portion 102, e.g., for performing installation and removal operations. In some such implementations, a cable-driving mechanism can further scale down (e.g., reduce) the overall size of the disclosed system.

Alternatively or additionally, the coupler 154 can be configured to detachably couple to the second body portion at any face-to-face angle between the base 155 of the coupler 154 and the second body portion 104. As used herein, the face-to-face angle means an angle in a plane perpendicular to opposing faces of the first and second body portions 102, 104. Thus, the attachment can be performed at any face-to-face angle (0-360 degrees) between the base 155 of the coupler 154 and the second body portion 104. In other words, the first and second body portions 102, 104 do not require any specific radial orientation relative to each other for coupling.

Alternatively or additionally, the coupler 154 can be configured to detachably couple to the second body portion 104 with minimal force. Optionally, detaching forces may be about 20 gram force unit, and attaching forces may be about 30 gram force unit. For example, in 15 tests performed using the leadless implantable device described herein, the detaching mean force of pull and push were 22.6 and 20.2, respectively, in gram force unit, while the attaching mean force of pull and push were 33.2 and 29.3, respectively, in gram force unit.

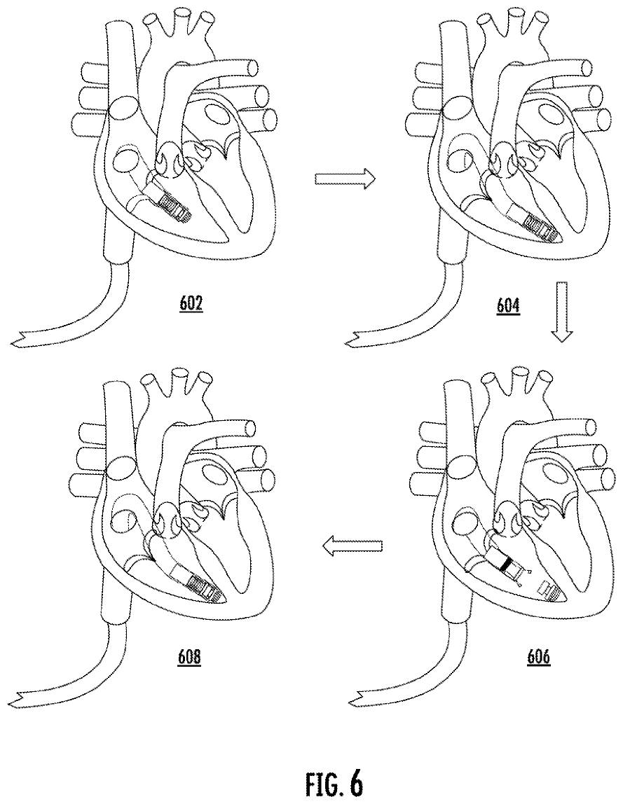

FIG. 6 illustrates an example method of implanting and removing the leadless implantable device shown in FIGS. 1A-1D. At step 602, the pacemaker main body (e.g., first body portion 102 shown in FIGS. 1A-1D) is illustrated, along with the fixation hook (e.g., second body portion 104 shown in FIGS. 1A-1D) being inserted inside the heart chamber with the help of a catheter. At step 604, the device is implanted in the heart. At step 606, the pacemaker main body (e.g., first body portion 102 shown in FIGS. 1A-1D) is removed, leaving the fixation hook (e.g., second body portion 104 shown in FIGS. 1A-1D) without damaging the surrounding tissue. At step 608, the new pacemaker main body (e.g., first body portion 102 shown in FIGS. 1A-1D) can be replaced by attaching it to the fixation hook (e.g., second body portion 104 shown in FIGS. 1A-1D).

It should be appreciated that the logical operations described herein with respect to the various figures may be implemented (1) as a sequence of computer implemented acts or program modules (i.e., software) running on a computing device (e.g., the computing device described in FIG. 7), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 7, an example computing device 700 upon which the methods described herein may be implemented is illustrated. It should be understood that the example computing device 700 is only one example of a suitable computing environment upon which the methods described herein may be implemented. Optionally, the computing device 700 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 700 typically includes at least one processing unit 706 and system memory 704. Depending on the exact configuration and type of computing device, system memory 704 may be volatile (such as random access memory (RAM)), non-volatile (such as read-only memory (ROM), flash memory, etc.), or some combination of the two. This most basic configuration is illustrated in FIG. 7 by dashed line 702. The processing unit 706 may be a standard programmable processor that performs arithmetic and logic operations necessary for operation of the computing device 700. The computing device 700 may also include a bus or other communication mechanism for communicating information among various components of the computing device 700.

Computing device 700 may have additional features/functionality. For example, computing device 700 may include additional storage such as removable storage 708 and non-removable storage 710 including, but not limited to, magnetic or optical disks or tapes. Computing device 700 may also contain network connection(s) 716 that allow the device to communicate with other devices. Computing device 700 may also have input device(s) 714 such as a keyboard, mouse, touch screen, etc. Output device(s) 712 such as a display, speakers, printer, etc. may also be included.

The additional devices may be connected to the bus in order to facilitate communication of data among the components of the computing device 700. All these devices are well known in the art and need not be discussed at length here.

The processing unit 706 may be configured to execute program code encoded in tangible, computer-readable media. Tangible, computer-readable media refers to any media that is capable of providing data that causes the computing device 700 (i.e., a machine) to operate in a particular fashion. Various computer-readable media may be utilized to provide instructions to the processing unit 706 for execution. Example tangible, computer-readable media may include, but is not limited to, volatile media, non-volatile media, removable media and non-removable media implemented in any method or technology for storage of information such as computer readable instructions, data structures, program modules or other data. System memory 704, removable storage 708, and non-removable storage 710 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 706 may execute program code stored in the system memory 704. For example, the bus may carry data to the system memory 704, from which the processing unit 706 receives and executes instructions. The data received by the system memory 704 may optionally be stored on the removable storage 708 or the non-removable storage 710 before or after execution by the processing unit 706.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Device Prototype

An implementation of the present disclosure is illustrated in FIG. 1A. For example, a leadless implantable device can include a DLPM body (also referred to herein as “first body portion 102”), a hook (also referred to herein as “second body portion 104”), and a control device 106 as shown in FIG. 1A. These can be designed with consideration of proper size and functionality. An electronic circuit for normal pulsing was designed, fabricated, and placed inside the DLPM body along with a 4.5V power supply battery. This disclosure contemplates that the leadless implantable device may include a power supply with a different voltage. For example, conventional pacemakers have battery voltage ranging from 0.1 to 15 V, depending upon the type and/or severity of the disease being treated. A 555 timer integrated circuit (IC) was used to generate a pulse with 10 percent duty cycle and 75 pulses per minute. This works as an example asynchronous pacemaker. The power supply battery voltage, 555 timer IC, and pulse characteristics described herein are intended only as non-limiting examples, and the present disclosure contemplates that the implementations of the present disclosure can include different types of electronic circuits (including circuits with different components that operate in different ways) for delivering different types of pulse waveforms (or any other purpose).

### Main DLPM Body

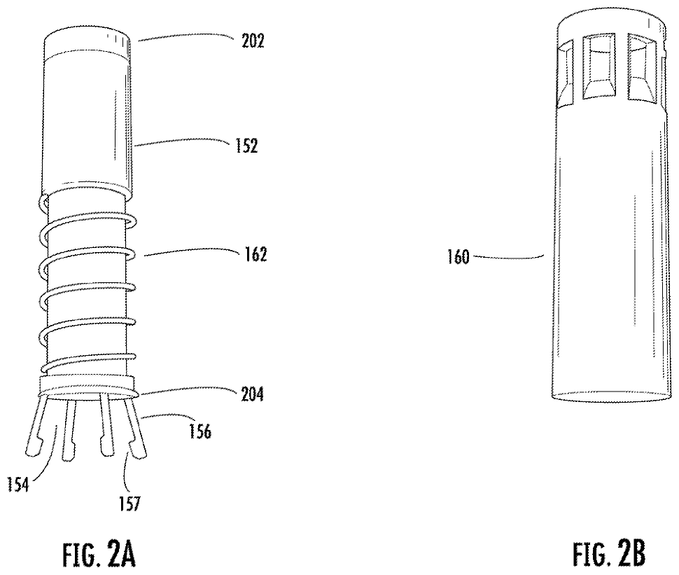

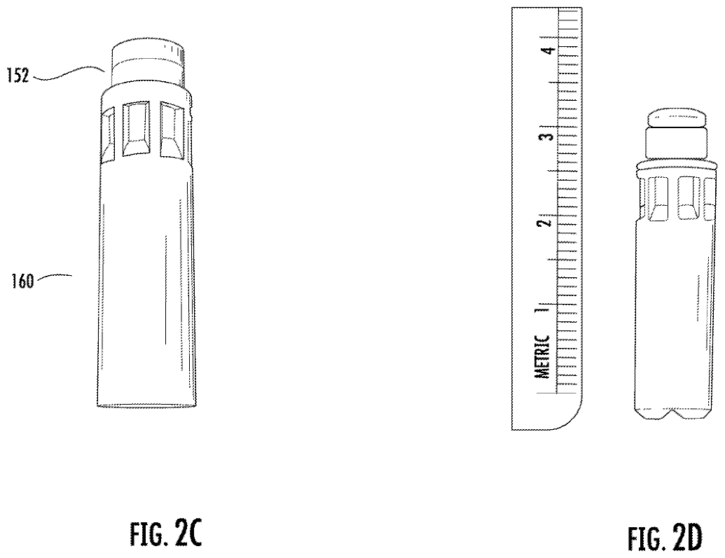

As illustrated in FIG. 2A, the main DLPM body can include an inner hollow capsule (also referred to herein as “housing 152”), claws (also referred to herein as “coupler 154”), spring 162, and electrodes 202, 204. In FIG. 2A, electrode 202 serves as a cathode and electrode 204 (which is also a coupler) serves as the anode. The inner hollow capsule encapsulates electronics and a battery inside. The cathode (electrode 202) is on the top end of the inner hollow capsule with electric connection to the electronics inside. The bottom end of the inner hollow capsule has expandable four electrically conductive claws radially distributed with connection to the circuit inside. The claws can be opened or closed by retracting or releasing an outer shell to achieve the goal of detaching and attaching with a hook. This claw can be a mechanical connector of the DLPM body with the hook and works also as a part of the anode (electrode 204). The DLPM body also includes an outer shell 160, and a rendered image of outer shell 160 is shown in FIG. 2B. The outer shell 160 can cover the inner hollow capsule locking a spring 162 in between them. In the relaxed case of the spring, the outer shell 160 will keep the claws closed, as in the rendered image of FIG. 2C and illustration of FIG. 2D, ensuring the strong connection between the DLPM body and the hook. But when the outer shell 160 is retracted, the spring 162 is compressed and claws open. This compressed spring provides force for closing the claws and keeps them closed. One end of this shell 160 has multiple grabbing points facilitating the control function by the control device. These grabbing points and the control device claws are designed in such a way that the grabbing process is orientation independent. At the end of the life of this pacemaker, only this DLPM body is detached from the hook and replaced with new pacemaker.

### Hook

The hook can be a metallic part with a spiral spring structure with a sharp needle end, which can be twisted to hook permanently into the myocardium of the heart chamber. The head of this part is made in such a geometry that allows us to attach the main part easily by launching onto it with almost 360-degree freedom of orientation. Claws (e.g. the coupler 154 illustrated in FIG. 2A) of the DLPM body connect with this hook and acts as the anode. Pulse signals are given to membranes of cardiac tissues through this hook which will alter the depolarization time correcting the pulse abnormalities. During the removal of the pacemaker, this hook can be left in place. In other words, the DLPM body can be detached from this hook and the new pacemaker is then launched onto this same old hook. This can ensure that there is reduced damage (or no damage) of the cardiac tissues during the extraction of the DLPM body.

### Controller

A specialized controller can be used for the implantation, removal and relaunching of the pacemaker. This controller can include a DC motor, a compact gear system, and control arms within a casing. The whole can be attached on the tip of a flexible delivery tube. With reference to FIGS. 3A-3D, a non-limiting example implementation of the control device of the present disclosure is shown. In these figures, the leadless implantable device includes a DLPM body (also referred to herein as “first body portion 102”), a hook (also referred to herein as “second body portion 104”), and a control device 106. In that implementation, the tube has a 9 mm outer diameter, which is comparable in diameter to the delivery sheath of 27 Fr outer diameter used by Medtronic [18] to attach the control device on the tip as a delivery system for the device implantation and replacement. In this example implementation, a DC motor of 7 mm diameter is used as torque source of mechanical actuation. A compact but powerful planetary gear as shown in FIG. 3A is doubly stacked for a gear ratio of 9:1 and used for torque multiplication [19]. This gear system does not only multiply the torque but also can slow down the high rotations per minute (rpm) of the DC motor, which can make the control more precise. The tip of this control device can have a fixed central base and radially distributed control arms capable of grab-pull and push-release action. Grab-pull action retracts the shell of the main part (i.e., DLPM body), and claws of the main part will open wide and makes the main part of the pacemaker ready to be detached while push-release action releases the shell, ensuring the attachment to the hook during the launching process. In FIG. 3B, the DLPM body is in a released condition; and in FIG. 3C, the DLPM body is grabbed by the control device's arm; and in FIG. 3D, the DLPM body is pulled back, making the arms wide open and completing the grab-pull action. In push-release action, this process takes place in reverse, first pushing the DLPM body down, closing the claws and releasing the DLPM by arms of the control device.

### Experimental Results

An experiment was conducted to demonstrate and test the working of the device. For this experiment, a heart with the comparable size of the human heart was printed using a resin 3D printer. Photopolymer resin used for the heart printing was not transparent, so an opening was made in the front part of the heart to show the internal processes of hooking the hook, detaching the pacemaker, and relaunching into the hook. A soft muscle-like part with silicon mold was placed on the bottom part of the right ventricle to mimic the myocardium of the heart where the leadless pacemakers are implanted.

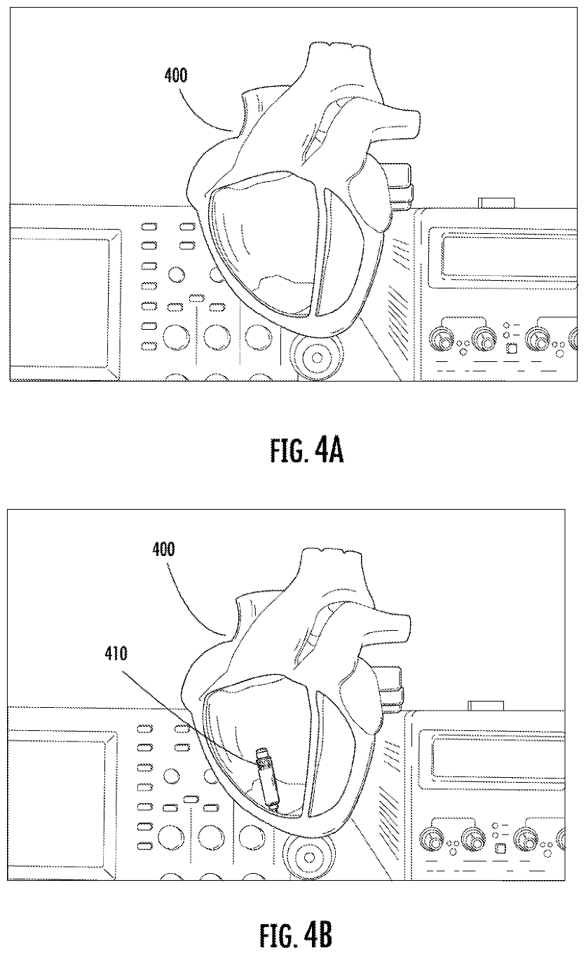

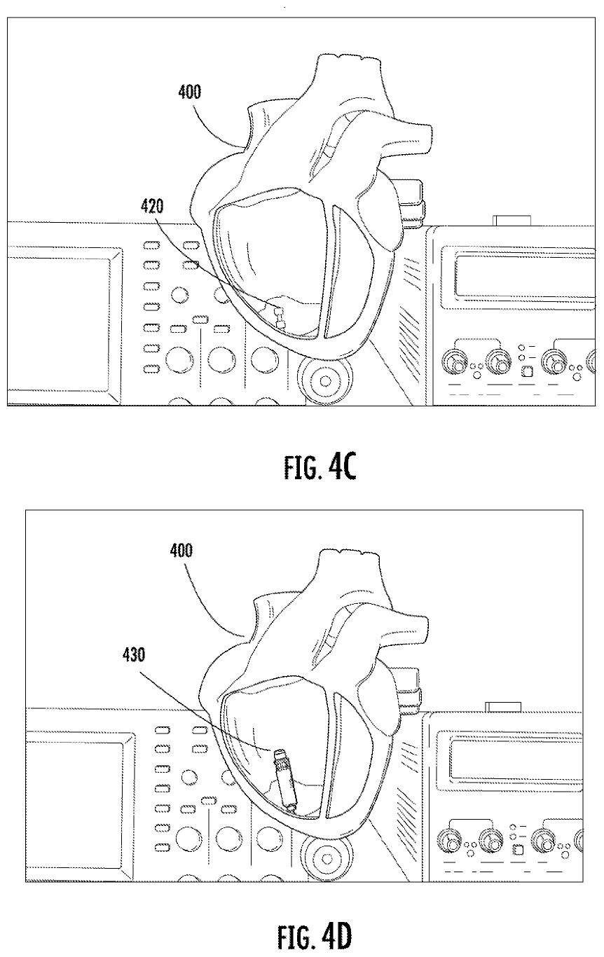

In the first experiment, the model 400 was placed in a stand in a vertical position as shown in FIG. 4A. The pacemaker 410 (e.g., first body portion 102 and second body portion 104 shown in FIGS. 3B-3D) was attached to the control device (e.g., control device 106 shown in FIGS. 3B-3D) and then introduced from the superior vena cava of the heart and passed through the right atrium and reached the right ventricle of the heart. After adjusting the position of the hook (e.g., second body portion 104 shown in FIGS. 3B-3D) on the proper place of implantation as shown in FIG. 4B, the control device was twisted to hook the spiral sharp end of the hook into the myocardium. After successfully implanting the pacemaker into the heart, the control device was detached from the pacemaker with push-release action. After making sure that the control device arm has released the pacemaker, the control device was retracted back and pulled out of the heart. Then, the connection of the DLPM with the heart was tested to determine whether it is being held strongly, by shaking the heart and by pulling the pacemaker. The position of the device after the test is shown in FIG. 4B.

In the second experiment, a test was performed showing the extraction of the old pacemaker from the heart and replacing a new pacemaker on the same old hook. For this, the control device was introduced in the same fashion as the implantation process but without the DLPM. The control device was guided into the heart chamber and the arms were released to make it ready to grab the DLPM. The old DLPM was detached from the hook with grab-pull action, and it was extracted out of the heart successfully. The hook 420 was left inside, hooked into the cardiac muscle is shown in FIG. 4C. The old DLPM (i.e., as shown in FIG. 4B) was detached from the control device and a new DLPM (i.e., as shown in FIG. 4D) was attached on the control device. The new DLPM was guided inside the heart chamber in same regular path and launched onto the hook by push-release action. The control device was retracted and pulled out of the heart. Proper attachment of the new DLPM on the same old hook was tested by shaking the heart and pulling the DLPM for a second time. The replaced pacemaker 430 (e.g., first body portion 102 and second body 104 shown in FIGS. 3B-3D) inside the heart after the second test is shown in FIG. 4D.

### Results and Discussion

The robustness of the connection between the DLPM and the hook can be verified by the strong attachment between them. This attachment was not broken or affected by multiple shake tests. Images taken before the replacement of the DLPM after the shake tests are shown in FIG. 4B where the DLPM is seen intact without any detachment or damage on the support silicon. After the replacement of the DLPM, again the condition of the DLPM was checked. The condition of the DLPM is shown in FIG. 4D, where no dislodgement or detachment of the DLPM and hook is visible. This evidence clearly proves that the connection between the DLPM and the hook is robust and could support the function of replacement of the DLPM multiple times without failure.

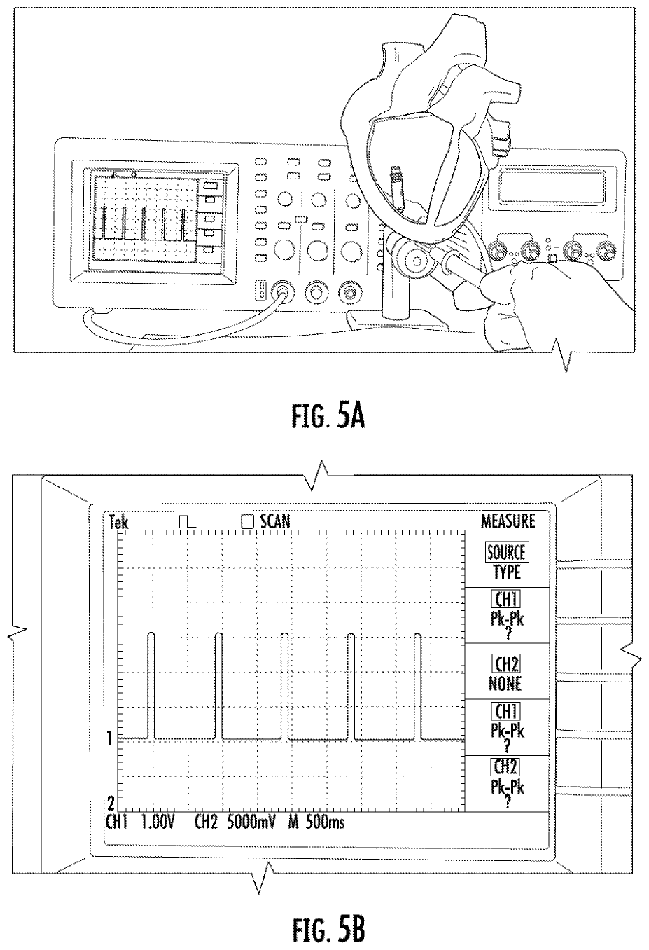

After the complete replacement of the DLPM, pulse signals generated by the DLPM were tested and verified to be working perfectly as shown in FIG. 5A. A pulse signal measured from an oscilloscope is shown in FIG. 5B.

Implementations of the present disclosure include a DLPM having the functionality of detachability. This functionality was successfully demonstrated in the tests of the example implementations described herein. The tests show that implementations of the present disclosure can be configured for implantation, detachment, and replacement of the DLPM in an artificial heart chamber. Additionally, implementations of the present disclosure include specific control devices that can be used to perform the process of detaching and attaching the DLPM to the hook very easily with the click of a switch. This development can help to reduce the failure of extraction of DLPM greatly and can make multiple LPM implantations very simple and/or safe.

### References

1. Roth, G. A.; Abate, D.; Abate, K. H.; Abay, S. M.; Abbafati, C.; Abbasi, N.; Abbastabar, H.; Abd-Allah, F.; Abdela, J.; Abdelalim, A., Global, regional, and national age-sex-specific mortality for 282 causes of death in 195 countries and territories, 1980-2017: a systematic analysis for the Global Burden of Disease Study 2017. The Lancet 2018, 392 (10159), 1736-1788.
2. Irisawa, H., Comparative physiology of the cardiac pacemaker mechanism. Physiological reviews 1978, 58 (2), 461-498.
3. Brown, H. F., Electrophysiology of the sinoatrial node. Physiological Reviews 1982, 62 (2), 505-530.
4. Ausubel, K.; Furman, S., The pacemaker syndrome. Annals of internal medicine 1985, 103 (3), 420-429.
5. Elmqvist, R. In senning A. Implantable pacemaker for the heart, Smythe NPD. Medical electronics: proceedings of the secondinternational conference on medical electronics, London: 1960.
6. Sanders, R. S.; Lee, M. T., Implantable pacemakers. Proceedings of the IEEE 1996, 84(3 ), 480-486.
7. Sperzel, J.; Burri, H.; Gras, D.; Tjong, F. V.; Knops, R. E.; Hindricks, G.; Steinwender, C.; Defaye, P., State of the art of leadless pacing. Ep Europace 2015, 17 (10), 1508-1513.
8. Ritter, P.; Duray, G. Z.; Zhang, S.; Narasimhan, C.; Soejima, K.; Omar, R.; Laager, V.; Stromberg, K.; Williams, E.; Reynolds, D., The rationale and design of the Micra Transcatheter Pacing Study: safety and efficacy of a novel miniaturized pacemaker. Ep Europace 2015, 17 (5), 807-813.
9. Seriwala, H. M.; Khan, M. S.; Munir, M. B.; Bin Riaz, I.; Riaz, H.; Saba, S.; Voigt, A. H., Leadless pacemakers: A new era in cardiac pacing. Journal of cardiology 2016, 67 (1), 1-5.
10. Ngo, L.; Nour, D.; Denman, R. A.; Walters, T. E.; Haqqani, H. M.; Woodman, R. J.; Ranasinghe, I., Safety and Efficacy of Leadless Pacemakers: A Systematic Review and Meta-Analysis. Journal of the American Heart Association 2021, 10 (13), e019212.
11. Middour, T. G.; Chen, J. H.; El-Chami, M. F., Leadless pacemakers: A review of current data and future directions. Progress in Cardiovascular Diseases 2021, 66, 61-69.
12. Kuang, R. J.; Pirakalathanan, J.; Lau, T.; Koh, D.; Kotschet, E.; Ko, B.; Lau, K. K., An up-to-date review of cardiac pacemakers and implantable cardioverter defibrillators. Journal of Medical Imaging and Radiation Oncology 2021.
13. Beurskens, N. E.; Tjong, F. V.; Knops, R. E., End-of-life management of leadless cardiac pacemaker therapy. Arrhythmia & electrophysiology review 2017, 6 (3), 129.
14. Bonner, M. D.; Neafus, N.; Byrd, C.; Schaerf, R.; Goode, L., Extraction of the Micra transcatheter pacemaker system. Heart Rhythm 2014, 11, S342.
15. Reddy, V. Y.; Miller, M. A.; Knops, R. E.; Neuzil, P.; Defaye, P.; Jung, W.; Doshi, R.; Castellani, M.; Strickberger, A.; Mead, R. H., Retrieval of the leadless cardiac pacemaker: a multicenter experience. Circulation: Arrhythmia and Electrophysiology 2016, 9 (12), e004626.
16. Dar, T.; Akella, K.; Murtaza, G.; Sharma, S.; Afzal, M. R.; Gopinathannair, R.; Augostini, R.; Hummel, J.; Lakkireddy, D., Comparison of the safety and efficacy of Nanostim and Micra transcatheter leadless pacemaker (LP) extractions: a multicenter experience. Journal of Interventional Cardiac Electrophysiology 2020, 57 (1), 133-140.
17. Zhang, J.; He, L.; Xing, Q.; Zhou, X.; Li, Y.; Zhang, L.; Lu, Y.; Tuerhong, Z.; Yang, X.; Tang, B., Evaluation of safety and feasibility of leadless pacemaker implantation following the removal of an infected pacemaker. Pacing and Clinical Electrophysiology 2021.
18. LAU, C. P.; LEE, K. L. F., Transcatheter leadless cardiac pacing in renal failure with limited venous access. Pacing and Clinical Electrophysiology 2016, 39 (11), 1281-1284.
19. Inalpolat, M.; Kahraman, A., A theoretical and experimental investigation of modulation sidebands of planetary gear sets. Journal of sound and vibration 2009, 323 (3-5), 677-696.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A leadless implantable device comprising:
a first body portion, the first body portion comprising:
a shell,
a housing configured to house at least one electrical component, the housing being arranged at least partially within the shell, and
a coupler attached to the housing; and
a second body portion configured to anchor to a surface of a patient's heart,
wherein the coupler comprises a base and a plurality of projecting members extending from the base, and wherein the coupler is configured to detachably couple to the second body portion.

2. The leadless implantable device of claim 1, wherein the coupler is configured to detachably couple to the second body portion at any face-to-face angle between the base of the coupler and the second body portion.

3. The leadless implantable device of claim wherein the coupler is configured to detachably couple to the second body portion with minimal force.

4. The leadless implantable device of claim 1, wherein the coupler comprises about five projecting members.

5. The leadless implantable device of claim 1, wherein each of the projecting members is an elongate member.

6. The leadless implantable device of claim 5, wherein the elongate member is about 5 millimeters (mm) long by 2 mm wide.

7. The leadless implantable device of claim 1, wherein the shell is moveable relative to the housing between a first position and a second position.

8. The leadless implantable device of claim 7, wherein the first body portion further comprises an actuation component configured to return the shell from the second position to the first position.

9. The leadless implantable device of claim 8, wherein the actuation component is a spring.

10. The leadless implantable device of claim 7, wherein the projecting members are configured to retract inside the shell when the shell is disposed in the first position.

11. The leadless implantable device of claim 7, wherein the projecting members are configured to extend outside of the shell when the shell is disposed in the second position.

12. The leadless implantable device of claim 11, wherein the projecting members are configured to extend beyond a perimeter of the shell when the shell is disposed in the second position.

13. The leadless implantable device of claim 1, wherein the coupler is configured to mechanically couple the first body portion to the second body portion.

14. The leadless implantable device of claim 13, wherein the shell is configured to prevent the coupler from mechanically uncoupling the first body portion from the second body portion when the shell is disposed in a first position.

15. The leadless implantable device of claim 1, wherein the coupler is configured to provide electrical coupling between the first and second body portions.

16. The leadless implantable device of claim 15, wherein a portion of the coupler is conductive and a portion of the second body portion is conductive.

17. The leadless implantable device of claim 16, wherein the conductive portion of the coupler is one or more of the projecting members.

18. The leadless implantable device of claim 16, wherein the conductive portion of the coupler is at least a portion of the base.

19. The leadless implantable device of claim 1, wherein each of the projecting members comprises a respective boss.

20. The leadless implantable device of claim 19, wherein each of the projecting members defines a respective first end and a respective second end opposite to the respective first end, wherein the respective first end is arranged in proximity to the base, and wherein the respective boss is arranged in proximity to the respective second end.

21. The leadless implantable device of claim 19, wherein the respective bosses are configured to engage with a corresponding void in the second body portion.

22. The leadless implantable device of claim 1, wherein each of the projecting members is formed from a plastic material having a conductive coating.

23. The leadless implantable device of claim 1, wherein each of the projecting members is formed from a metal.

24. The leadless implantable device of claim 1, wherein the at least one electrical component comprises a power source, a pulse generator configured to deliver electrical stimulation, or a controller comprising a processor and a memory.

25. The leadless implantable device of claim 1, wherein one or more of the projecting members is a flexible projecting member.

26. A system comprising:
the leadless implantable device according to claim 1, and
a control device configured to mechanically actuate the first body portion of the leadless implantable device.

27. The system of claim 26, wherein the control device comprises:
a motor;
a gear system mechanically coupled to the motor; and
a plurality of control arms mechanically coupled to the gear system, wherein the control arms are configured to detachably couple to the shell of the first body portion of the leadless implantable device.

28-50. (canceled)
